(19) United States
(12) Patent Application Publication
(10) Pub. No.: US 2011/0093084 A1
(43) Pub. Date: Apr. 21, 2011

(54) ARTIFICIAL JOINT PRESERVING TENDON AND/OR SESAMOID BONE STRUCTURE

(76) Inventor: Troy N. Morton, Red Springs, NC (US)

(21) Appl. No.: 12/908,835

(22) Filed: Oct. 20, 2010

Related U.S. Application Data
(63) Continuation-in-part of application No. 12/027,590, filed on Feb. 7, 2008.
(60) Provisional application No. 60/889,195, filed on Feb. 9, 2007.

(51) Int. Cl. A61F 2/42; A61F 2/30; A61B 17/00

(52) U.S. Cl. 623/21.19; 623/18.11; 606/86 R

(57) ABSTRACT

An artificial toe joint utilizes a ball in socket joint structure and arms or side walls which are exterior to the bone, the side walls extending around depressions in the metatarsal bone or phalangeal bone, respectively so as to not interfere with sesamoid bones or the attachment of tendons. The resulting joint provides improved strength and durability, and may be used to repair joints which are not suitable for installation of a prior art artificial joint.

### RELATED APPLICATIONS

The application claims the benefit of U.S. Provisional Patent Application Ser. No. 60/889,195, filed Feb. 9, 2007, and U.S. patent application Ser. No. 12/027,590, filed Feb. 7, 2008, which are incorporated herein in their entirety by reference.

### BACKGROUND OF THE INVENTION

1. The Field of the Invention

The present invention relates to an improved artificial joint. More specifically, the present invention relates to an artificial joint providing increased durability, reduced damage to the surrounding bone and which may accommodate the sesamoid bones and/or tendons associated with the joint.

2. State of the Art

Toe joints such as the proximal metatarsal phalangeal joint, the proximal most toe joint of the foot, may become damaged from injury, etc. and may then be replaced. As a toe joint is damaged and deteriorates, symptoms may include loss of propulsion, transfer lesions, metatarsalgia (pain and inflammation of the ball of the foot), gait alterations, pain, etc. Indicators for joint replacement include: hallux limitus, hallux rigidus, hallux abducto valgus, rheumatoid arthritis, osteoarthritis, previous surgery at the joint which is painful or which resulted in an instable joint, joint problems after a prior joint surgery, failed joint surgery, etc.

Currently, artificial metatarsal joints exist which are implanted by cutting off the ends of the bones which form the natural joint (typically the metatarsal phalangeal joint(MTPJ)), reaming out the cut ends of the bones (the base of the proximal phalanx and the head of the metatarsal) to receive the stems of the artificial joint, and inserting the artificial joint. The prior art artificial joints place stress on the bone surrounding the joint, often resulting in destruction of the bone surrounding the joint and thus failure of the artificial joint. It is common for artificial toe joints to fail about five years after replacement. Once the joint has failed, the bone structure surrounding the joint (the cut end and hole into which the artificial joint has been inserted) has often degraded to where the joint must be fused together. It is easily appreciated that a fused toe joint is highly undesirable as it limits mobility, and may make it significantly harder for a person to accomplish daily tasks such as walking.

Coring out of the proximal end of the phalangeal bone can also interfere with the attachment of the flexor hallucis brevis to the phalange in the big toe. Thus, some artificial joints provide artificial sesamoid bones to replace those found in the flexor hallucis brevis. See e.g. Wyss et al., U.S. Pat. No. 4,787,908. Thus, while the joints can weaken the bone structure itself, they can also complicate the existing tendon structure associated with a joint which helps it move as desired. Additionally, it is important to note that current joints generally do not provide compensation for angular deviations at the first metatarsal phalangeal joint. Such deviations include, but are not limited to: hallux abductovalgus, inter phalageous angle, plantarflexed as well as dorsiflexed metatarsal head, intermetatarsal angles, and proximal articular set angle as well as the distal articular set angle. The advent of a total implant that can compensate for such deviations is very advantageous secondary to angular correction. These corrections will allow reduction of pain proximally in the foot as well as extend the life of the implant.

It is desirable that an artificial toe joint should achieve certain results. The artificial joint should be stable and provide stability to the patient, such as when standing and walking. The artificial joint should also provide a relatively pain free range of motion to the patient. The artificial joint should allow the patient to walk and move in a natural manner. It is desirable that installation of an artificial joint provide an increase in activity levels and an improvement in the lifestyle of the person. An artificial joint should provide long term success; promoting the strength of the surrounding bone and resisting deterioration of the resulting joint so as to minimize the need for the later fusing of the joint.

There is thus a need for an artificial toe joint which overcomes some or all of the limitations of available artificial toe joints. Specifically, there is a need for an artificial toe joint which has less effect on the bone structure around the joint. It is also desirable to provide an artificial joint which allows for angular deviation correction to reduce stress and strain proximally in the foot. It is also desirable to provide resurfacing of the existing anatomy which is low profile and anatomically similar to existing structures. Thus, there is a need for an artificial toe joint which provides greater long term success of the artificial joint and which replicates existing anatomical motion. There is also a need for an artificial toe joint which may be used in replacing a previously installed artificial toe joint which has failed to thereby eliminate the need to fuse the joint. There is a further need for an artificial toe joint which is easier to install. It will be appreciated that achieving any one of these will be an improvement in artificial toe joints, while achieving multiple of these ends would constitute a substantial improvement for patients.

### SUMMARY OF THE INVENTION

It is an object of the present invention to provide an improved artificial toe joint.

According to one aspect of the invention, an improved artificial toe joint is provided which is suited for a first metatarsal phalangeal joint replacement, but may be used for other toe joints as well. Ideally, artificial joints made according to the present invention can be used for the interphalangeal joints of the first digit, the interphalangeal joints of the lesser digits, as well as the metatarsal phalangeal joints of the lesser digits.

According to one aspect of the present invention, an artificial toe joint is provided which encases the ends of the bones adjacent the joint. Such a configuration may be advantageous in that it reduces or eliminates the need to core out the distal end of the bone. This may also be advantageous where it leaves existing structures such as tendons and sesamoid bones undisturbed or less disturbed than currently used artificial toe joints. By enclosing the ends of the bones adjacent the joint, the artificial joint results in a more stable structure and reduces the stress on the ends of the bone by distributing the stresses along the bone, promoting improved long term success for the artificial joint. By leaving tendons and sesamoid bone, the impact on the existing structure may be reduced and the joint may be provided with more normal movement.

In accordance with another aspect of the present invention, a resurfacing technique is provided. This resurfacing is accomplished through the advent of a low profile joint cutter that contours the existing anatomical structures providing minimal bony disruption, placing the articular surface in alignment thus reducing any existing angular deviations, providing anatomical motion, and reducing pain.

In accordance with another aspect of the present invention, an artificial joint is configured to compensate for metatarsal length variations, which is typically not available in currently used artificial toe joints. The present invention may include an articulating surface that can be varied in thickness to compensate for first ray length. When such variances are considered and addressed the resultant mobile and rectus first ray will provide for pain reduction both distally and proximally to the implanted surface.

In accordance with another aspect of the present invention, an articulating surface of the joint may include a cut-out that minimizes effects on other existing structures such as tendons or sesamoid bones. Moreover the articulating surface may be configured to compensate for radial displacement or misalignment of the joint and/or the toe.

According to another aspect of the present invention, enclosing the ends of the bone surrounding the artificial joint may allow the present artificial toe joint to be installed in place of previously installed artificial toe joints which have failed. Such an installation may eliminate the need to fuse the joint. Additionally, the artificial joint of the present invention makes possible the modification of existing fused joints.

In accordance with still yet another aspect of the invention, an artificial toe joint may be applied to joints including, but not limited to the inter-phalangeal joints of all lesser digits, as well as the metatarsal phalangeal joints of all lesser digits. It is appreciated that the fusion of these joints does not result in the same loss of mobility and the same degree of detriment to a patient as does the fusion of the proximal metatarsal phalangeal joint, but does result in some detrimental effects for the patient. For example, the second metatarsal phalangeal joint can develop Freiberg's infraction resulting in metatarsal head deformation and loss of cartilage. The treatments are generally limited following development of Freiberg's. The present invention, however, is believed to reduce such risks.

Furthermore, the invention is not restricted to the metatarsal phalangeal joint of the first digit and can be utilized to replace the inter-phalangeal joint of the first digit which too is often damaged secondary to cartilage loss and is often fused. As will be explained below, the present invention is suitable for both conditions as it allows resurfacing of the joint.

These and other aspects of the present invention are realized in an artificial joint as shown and described in the following figures and related description. It will be appreciated that while the invention includes numerous aspects, any individual embodiment need not accomplish all aspects of the invention and the invention should be determined by the appended claims.

### BRIEF DESCRIPTION OF THE DRAWINGS

Various embodiments of the present invention are shown and described in reference to the numbered drawings wherein:

FIG. 1 shows a perspective view of a first metatarsal bone and a proximal phalangeal bone and sesamoid bones which comprise the first metatarsal phalangeal toe joint in a normal foot;

FIG. 2 shows the toe joint of FIG. 1 cut to receive a prior art artificial joint;

FIG. 3 shows a perspective view of the back side of one piece of an artificial joint of the present invention;

FIG. 4 shows a perspective view of a back side of an artificial joint member formed in accordance with the principles of the present invention which could be used on a metatarsal bone;

FIG. 5 shows a perspective view of a back side of an artificial joint member which could be used on a phalangeal bone;

FIG. 6 shows the toe joint of FIG. 1 cut to receive the artificial joint of the present invention;

FIG. 7 shows the toe joint of FIG. 1 having an artificial joint made in accordance with the present invention installed thereon;

FIG. 8 shows another view of the toe joint of FIG. 1 having an artificial joint of the present invention installed thereon;

FIG. 9 shows another view of the toe joint of FIG. 1 having an artificial joint made in accordance with the present invention installed thereon;

FIG. 10 shows another perspective view of the toe joint of FIG. 1 having an artificial joint of the present invention installed thereon;

FIG. 11 shows a bottom perspective view of the artificial joint of FIG. 10;

FIG. 12 shows a perspective view of a foot with the artificial joint of FIG. 10 installed thereon;

FIG. 13 shows a perspective view of a metatarsal bone and proximal phalanx bone forming a toe joint;

FIG. 14 shows the bones shown in FIG. 13 having a dorsal cut formed on the metatarsal bone;

FIG. 15 shows the toe joint of FIGS. 13 and 14 and a template used for making cuts on the toe joint;

FIG. 16 shows a proximal phalanx bone artificial joint member and an implant in accordance with one aspect of the present invention;

FIG. 17 shows a cross-sectional view of the artificial joint member and implant of FIG. 16;

FIG. 18 shows an exploded view of the artificial joint with an implant;

FIG. 19 shows an alternate exploded view of the artificial joint with implant of FIG. 18;

FIG. 20 shows a phalangeal bone with artificial joint member and a selection of inserts;

FIG. 20A shows cross-sectional views of the inserts of FIG. 20;

FIG. 21 shows a bit used for implant surface preparation on the bone; and

FIGS. 22 and 23 show side views of the preparation of the metatarsal and proximal phalanx using the bit of FIG. 21.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims.

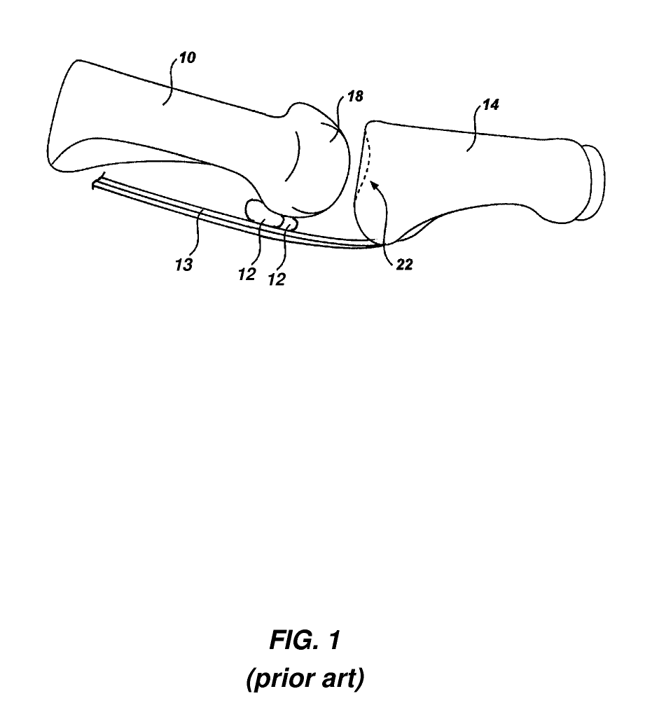

Turning now to FIG. 1, a perspective view of the bones of the proximal toe joint (the metatarsal phalangeal joint) is shown. In particular, the joint of the first metatarsal phalangeal joint (i.e., “big toe”) is shown.

The joint includes a metatarsal bone 10 and a phalangeal bone 14. The metatarsal bone 10 includes a rounded end 18 which, together with a depression 22 in the phalangeal bone 14 and the associated cartilage and tissue, forms the joint. In the case of the first metatarsal phalangeal joint (big toe joint), two sesamoid bones 12 ride within depressions at the bottom of the metatarsal bone and close to the metatarsal phalangeal joint. The sesamoidal bones 12 are disposed in the tendon of the flexor hallucis brevis 13 which moves the toe downwardly in response to muscle contractions in the foot. The replacement of the toe joint may be necessitated by damage to the bones 10, 14 or to the cartilage and tissues of the joint. (While described in context of the first metatarsal phalangeal joint, the same anatomical presentations exist throughout the lesser metatarsal phalangeal joints with the exception of size and the absence of the sesamoid bones. Additionally, the inter-phalangeal joints also minor somewhat the metatarsal phalangeal joints and it will be appreciated that the present invention could be applied therein if desired.)

Historically, the sesamoid apparatus formed by the sesamoid bones 12 have not received significant attention in its role in the successful surgical outcome of bunion or arthritic surgeries of the metatarsal phalangeal joint. As such, little attention appears to be given this apparatus in the current implant market. However, the implant of the present invention takes measures to leave this portion of the joint unadulterated postoperatively. In accordance with one aspect of the present invention, it is anticipated that leaving the sesamoid apparatus intact will lead to a success and tolerance level that is not seen in current artificial toe joints. The sesamoid apparatus in nature serves as a gliding surface to facilitate gliding of the metatarsal head on a weight bearing surface of the sesamoid. In accordance with one aspect of the present invention, the implant is purposely configured to maintain the active gliding surface, thereby further maintaining the normal anatomical sesamoid/joint function.

Additionally, in regard to intrinsic flexor, current devices actually release the intrinsic flexors following which they either are left unattached or have to have additional procedural techniques to reattach these structures intraoperatively. Many implants do not even replicate the natural function of the metatarsal phalangeal joint (MTPJ), but serve purely as function spacer/hinges. The designs of some embodiments of the present invention incorporate variations which allow the intrinsic anatomical tendon and ligament attachments to remain intact. This serves to decrease the amount of post operative contracture and scarring thus restoring normal anatomical function of the joint. In addition, complications in the distal portion of the joint are addressed by preventing the release of the pericapsular structures. Again, this may limit the amount of postoperative scarring, maintaining the natural insertions of the flexor attachments and avoids the historical flail toe deformity of MTPJ surgery. Applicant is unaware of other implants that address this problem adequately.

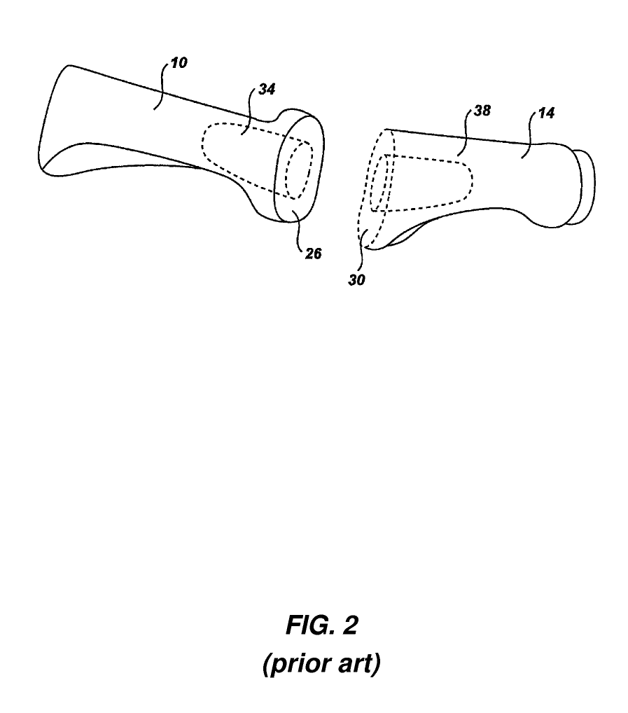

FIG. 2 shows the bones 10, 14 of FIG. 1. The bones 10, 14 have been prepared for an artificial toe joint of the prior art. The ends of the bones 10, 14 have been cut flat as indicated at 26, 30. Additionally, holes 34, 38 have been formed in the ends of the bones 10, 14 to receive the artificial toe joint. In other words, the ends of the bones 10 and 14 have been cored out. Some prior art artificial joints have posts on both ends which are fused into the holes 34, 38 to attach the joint to the bones 10, 14. A flexible member, not shown, connects the posts to form the artificial toe joint and to allow movement of the phalangeal bone 14 relative to the metatarsal bone 10.

A problem with the prior art artificial joints is that the strength and structure of the bone is compromised by placement of the artificial joint. The toe bones 10, 14 are fairly small, and their strength may be significantly compromised by drilling or coring out the bones. The posts of the artificial joint combined with the hollowed out bones 10, 14 result in places of high stress and typically result in damage to the bones. Once the bone is damaged, it may be difficult to repair the damage and install another artificial joint. When an artificial joint fails, the bones 10, 14 must often be fused together.

Similarly, when repairing joints, the existing structure may be damaged or replaced. In some prior art treatments, the tendons may be detached from the phalange bone and/or the sesamoids removed or replaced with artificial sesamoids. An example of such is shown in U.S. Pat. No. 4,787,908 (Wyss et al.). Each disturbance of the structure may require further healing and potentially adaptation to surgical implants as well as a potential point of failure in the artificial joint.

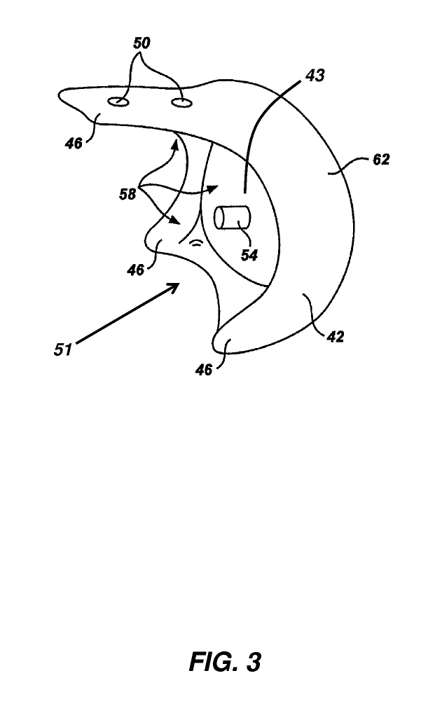

FIG. 3 shows a perspective view of the back side of an artificial joint member in accordance with the present invention. The artificial joint member 42 shown replaces the rounded end 18 of the metatarsal bone 10. The artificial joint member 42 may include a generally concave or recessed portion 43 which receives an end of a bone, and support members in the form appendages (i.e. arms, collars, etc.) which extend around the end of the bone and extend along the exterior of the bone along its outer length to provide for an attachment mechanism. (The support members may extend from at or adjacent a peripheral edge between the first and second sides of the support members and extend so that a concave receptacle is formed between the support members and the second side of the implant where it receives the end of the bone even if the actual face on the second side is flat or somewhat convex. The enables the end and a portion of the top and/or side of the bone to be covered by the implant).

As shown in FIG. 3, the appendages or support members are formed by arms 46. One or more of the arms 46 may include holes 50 for receiving a pin or screw to further secure the artificial joint member 42 to the bone.

The arm or arms 46 typically extend from the periphery of the main body of the artificial joint member and extend from the top side, lateral side(s) and/or bottom side, although the top side is most common. This allows the arm(s) 46 to extend along the outside of the bone rather than relying on a cored out portion of the bone for support. This in turn, is believed to allow the artificial joint member to cause less damage to the bone, to allow the joint member to be used for a longer period of time, and decrease the risk of fusion or other similar remedies in the event of joint failure.

The artificial joint member may also include cut-outs 51 that avoid covering a portion of the bone which may be associated with other structures. In FIG. 3, the cut-out 51 may be formed to avoid covering the depressions in which the sesamoids are present. The bottom thickness and length may also be adjusted to prevent relocation of the flexor hallucis brevis tendon associated with the sesamoid bones. In other embodiments, the end may consist of arms 46 on the sides and/or top of the bone with the cut-out 51 at the bottom.

The artificial joint member 42 may also include a small post or spike 54 to aid in securing the artificial joint member to the bone. The post or spike 54 is typically small compared to the size of the bone and does not compromise the strength of the bone similar to the holes formed in prior art devices. The post or spike 54 can help keep the artificial joint member 42 from sliding back and forth across the front of the bone as the bone heals and attaches itself to the artificial joint member, and to provide additional support once healing is complete.

The inside surfaces of the artificial joint member 42, as indicated at 58, may be made somewhat rough or porous, and may be coated with compounds which promote the adhesion of the bone to the artificial joint member. These compounds may be bone growth compounds and other compounds known in the art. The artificial joint member 42 may thus be initially held in place by the post or spike 42 and pins or screws 86, and then may become further attached to the bone as the bone grows and affixes itself to the inside surface 58 of the artificial joint member.

The artificial joint is formed by the first artificial joint member 42 and a second artificial joint member (78 of FIG. 5-7) which may also have a recess 79 configured to receive all or part of the end of a bone. The back side of artificial joint member 78 (which is attached to bone 14) may be somewhat similar to that of artificial bone member 42, having arms 46 or the like extending from the top, bottom and/or lateral sides so as to help define the recess 79 and extending for attachment to the outside of the bone 14, a post or spike 54, holes 50 for pins or screws 86, a compound for promoting adhesion to the bone, etc. Artificial joint member 42 is formed with a rounded end 62, similar to the rounded end 18 of bone 10. Artificial joint member 78 is formed with a recess similar to the recess 22 of bone 14. Both joints may also have a cut-out or shortened sidewall on a bottom portion to prevent interference with the sesamoid bones and associated tendons, respectively. Thus, the artificial joint recreates the natural joint, promoting a natural motion and use of the joint.

Like the inside surfaces of the artificial joint member, the arms 46 may be coated with material, such as bone morphogenic protein, to facilitate attachment to the bone. In one preferred embodiment, the dorsal arm 46 may be at least somewhat porous and coated on both inside and outside surfaces with bone growth enhancing material.

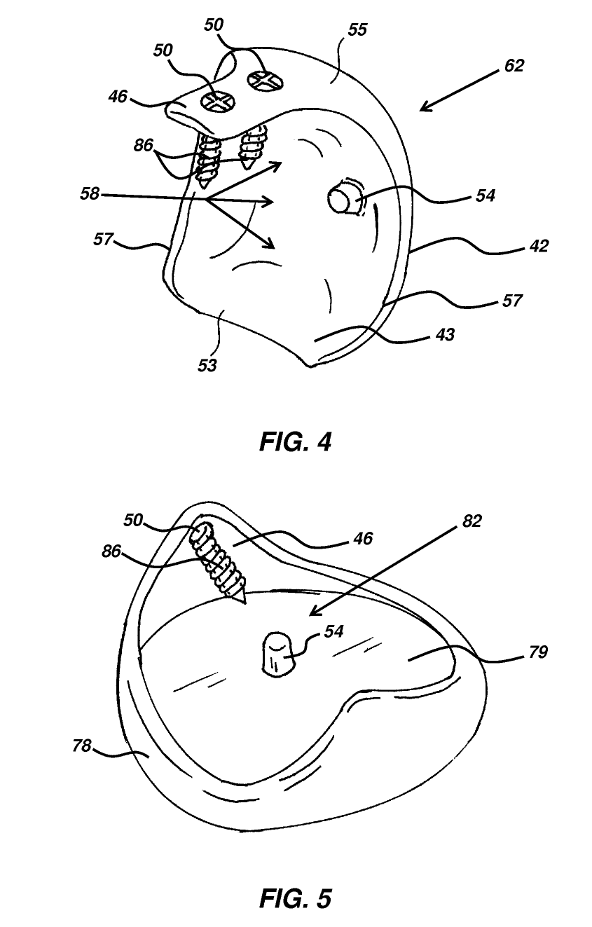

Turning now to FIG. 4, a perspective view of the back side of a metatarsal artificial joint member 42 is shown. In one embodiment, instead of a cut-out 51, a bottom sidewall 53 may not extend far enough from the recess 43 to interfere with the sesamoid depressions. The top sidewall 55 and side sidewalls 57 may extend around the bone on the top and/or sides, but remain open at the bottom. In another embodiment, the sidewalls 57 extend similarly along the bone with an arm projecting from one or more sides for attachment. In some embodiments, the end may be collar-shaped with a cut-out 51 at the bottom. Regardless of the specific combination, the artificial joint member 42 is designed to cover the end of the bone (or the cut end of the bone), rather than being inserted (other than the small post 54) into a cored out bone. Thus, the back or second side of the artificial joint member 42 is generally concave for receipt around the bone.

Turning now to FIG. 5, a perspective view of the back side of an artificial joint member 78 which is likely to be used with a phalangeal bone is shown. The artificial joint member 78 may be configured with a depression or void 82 to form a generally concave structure which receives the end of the phalangeal bone and may include a depression or void (not shown in FIG. 5) to match a metatarsal artificial joint member or an insert which engages a metatarsal artificial joint member.

A spike 54 and an arm 46 with a hole 50 for a screw 86 may aid in the placement and fastening of the phalangeal artificial joint member 78 to the phalangeal bone, just as arm 46, hole(s) 50 and screw(s) 86 may be used on the metatarsal artificial joint member 42 shown in FIG. 4.

The process for placing the artificial toe joint may include the use of a pre-formed template system. The template will be placed intra articular and serve as a guide for shaping the head of the metatarsal as well as the base of the proximal phalanx. The template will serve as a guide for placement of the sagital saw blade or other cutting device, thus reducing surgical error and allowing for ease of placement of the joint. The template will cause the corresponding bony surface to minor the inside of the articulated resurfacing implant, while preferably avoiding damage to the tendon or sesamoid bones. The template system may vary in size to compensate for variance in bony girth. The template used may correlate numerically with the implant that will be placed, thus improving surgical success as well as implant longevity, with minimal interference with the tendon and sesamoid bones if such can be accomplished.

While the bones may be predrilled to receive posts in the artificial joint, it is believed that pre-drilling is not required, as the posts can be tapped into the bone with a mallet. The posts may vary in length, but may remain relatively small to facilitate placement and securing of the implant so that it does not move while causing minimal disruption to the interior of the bone. This is in contrast to prior art artificial toes in which the portion of the artificial joint is inserted into the bone and risks further damage to the bone.

A flexible member or other insert, discussed in more detail below, may be placed after the implant has been positioned. Once in place, the length of the first ray may be determined using removable and reusable guides. Once the thickness and type of insert has been determined, the actual insert disk can be opened and placed in the artificial joint member with the appropriate void. This may be accomplished using a tongue and groove system that can be modified, using a set screw or by other fastening means. Following placement, the first digit can be placed through range of motion to ensure correction and placement suitability. By selecting a disk or other insert having the desired curvature orientation, etc., a physician is able to determine toe alignment and can help correct misalignments which inhibit proper use of the joint and may have contributed to the need to an artificial joint in the first place.

It has been found that it is preferable for the template system to produce a generally mirrored surface on the metatarsal (etc.) head to that of the implant. This can be done with minimal bone reconstruction and with the use of the previously described template system. Thus, the implant essentially becomes a resurfaced metatarsal head, functioning in the same manner as the original prior to damage (or similar to a typical undamaged metatarsal bone). By avoiding interference with the sesamoid bones and related tendons, the joint may function very similarly as before.

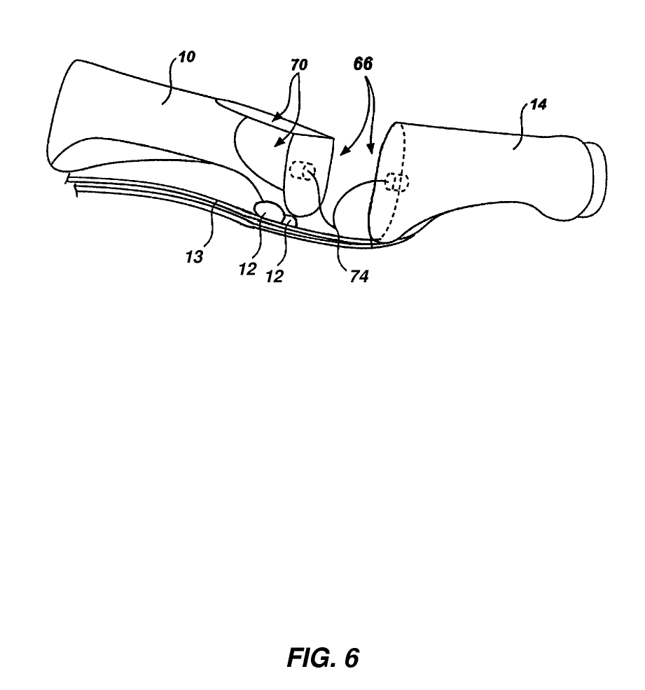

FIG. 6 shows the toe bones 10, 14 of FIG. 1 as having been cut and prepared for an artificial joint of the present invention. The adjoining ends of the bones 10, 14 have been cut off as indicated at 66. (While shown as a fairly linear cut, it will be appreciated that the end may be shaped as desired). Additionally, the top, bottom, or sides of the bones 10, 14 may be cut as indicated at 70 to provide sufficient room for the artificial joint member and to create sufficient attachment surfaces for the same while keeping the profile of the artificial joint member to a minimum. Additionally, a small hole or recess 74 may be formed in the ends of the bone 10, 14 if desired to receive any post or spike (54 of FIGS. 3-5) which may be utilized in the artificial joint members. It will be appreciated that the hole 74 may not be necessary if the post or spike 54 is not used, or is small enough and appropriately shaped to be simply pressed into the bone 10, 14. (It will be appreciated that other processes of bone preparation may be used as will be explained below). Moreover, the preparation may be performed without significant disturbance of the sesamoid bones or their associated tendon.

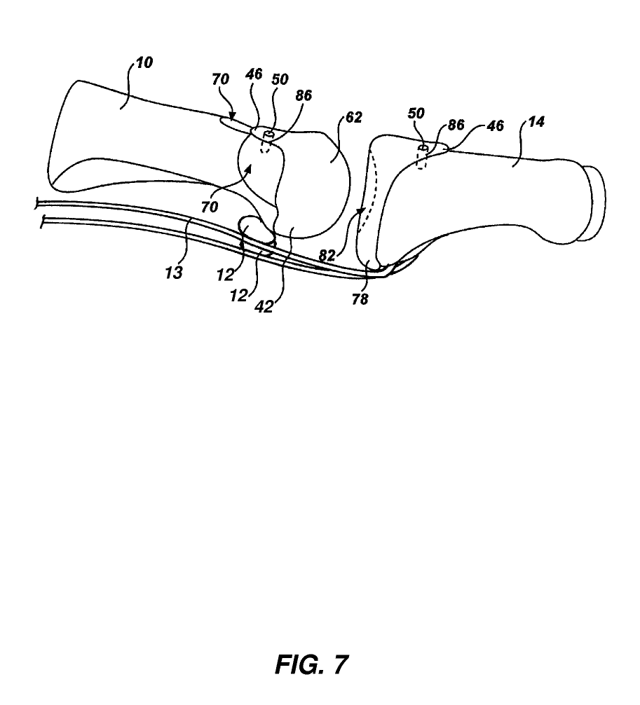

FIG. 7 shows the first and second artificial joint members 42, 78 of the present invention attached to the toe bones 10, 14. The rounded end 62 disposed on the front or first side of the artificial joint member 42 is similar in shape and size as the end 18 of the natural metatarsal bone 10. The arm 46 (or a collar, etc) extends from the back or second side of the artificial joint member and along the exterior of the bone and thereby holds the rounded end securely to the bone without placing stress on the interior of the bone as is done in many commonly used artificial toe joints.

Artificial joint member 78 is formed with a recess 82 in the front or first side which is generally rounded and concave to receive the rounded end 62, and to be similar in shape and function to the recess 22 found naturally in the phalangeal bone 14. One or more arms 46 (or a collar, etc) extend from the back or second side to hold the second artificial toe joint member 78 to the exterior of the phalangeal bone 14.

Pins or screws 86 may be inserted through the holes 50 in the artificial joint members 42, 78 and into the bones 10, 14 to secure the artificial joint members. As has been discussed, the insides of the artificial joint members may be formed with a texture or coated with a bone growth promoter to stimulate the bones 10, 14 to adhere to the artificial joint members.

The number and placement of arms 46, pins, screws 86 or sidewalls may be adjusted to compensate for the cut-out or a shortened sidewall. Screws 86 or pins on the side and/or top of the bone may provide enough holding force to prevent undesired movement of the artificial joint members. Arms or sidewalls 46 extending along the top or sides of the bone may be extended to provide a longer lever with which to aid in the sustained attachment of the artificial joint member.

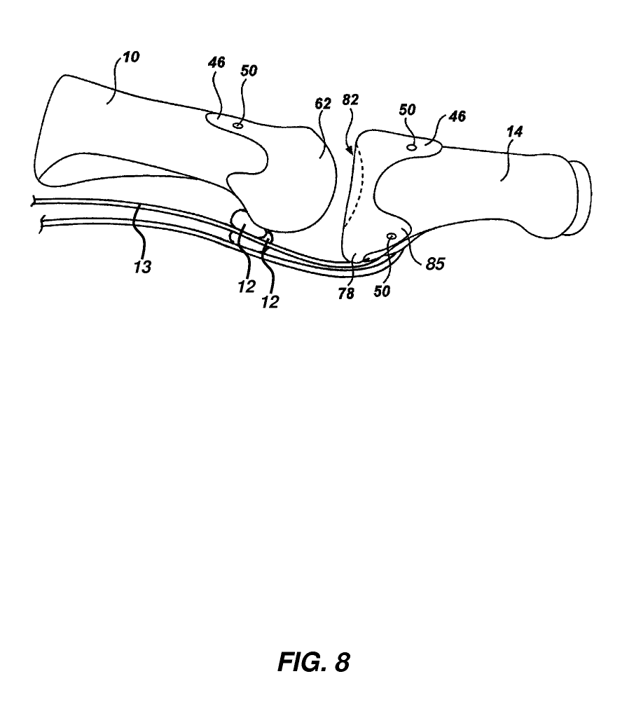

FIG. 8 shows an alternate configuration of the artificial joint of the present invention. The artificial joint members 42, 78 are similar to those of FIG. 7 but include additional arms 46. FIG. 7 shows one larger arm 46 at the top of the artificial joint members 42, 78. FIG. 8 shows additional arms 46 on the sides of the artificial joint members 42, 78, and may include additional holes 50 for pins or screws 86 to secure the artificial joint members to the bones 10, 14. Other attachment means could also be used. The artificial joint members 42, 78 may also contain the other structures discussed with respect to FIGS. 3-7 which are not shown here for clarity.

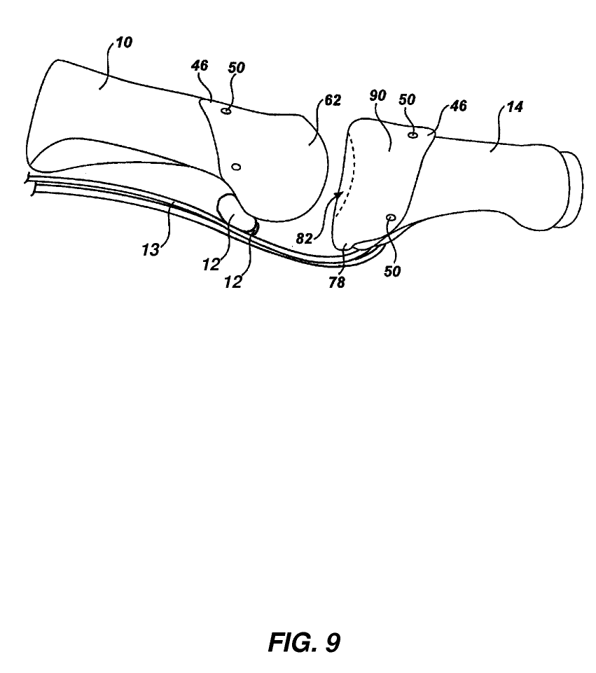

FIG. 9 similarly shows an alternate configuration of an artificial joint of the present invention. The artificial joint members 42, 78 are formed with elongated side walls 90 which extend around a much larger portion of the bones 10, 14; such as extending around the tops and sides of the bones. The side walls 90 may form a collar which wraps around the end of the bone 10, 14, but leaves the bottom relatively unaltered. The artificial joint members 42, 78 may also be formed with arms 46 which extend further than the side walls 90, and may include one or more holes 50 for receiving pins or screws 86 to affix the artificial joint members to the bone 10, 14. The artificial joint members 42, 78 also include the remaining structures shown in FIGS. 3-8 but which are not shown for clarity.

In viewing FIGS. 7-9, it can be appreciated that the number and relative size of arms 46 may be varied. Additionally, the use of side walls 90 which extend around a more substantial portion of the bones 10, 14 may be varied. Providing more arms or longer arms, or using side walls 90 may make the artificial joint members 42, 78 more difficult to install, or make each particular size of artificial joint member fit a more limited size of toe bones 10, 14. However, providing more or larger arms 46 or side walls 90 may achieve a stronger bond to the bones 10, 14 and result in a stronger artificial joint which may be more durable and last longer.

Additionally, it may be possible to repair damaged bones 10, 14 by using more arms 46 or side walls 90. As has been mentioned, installation of prior art artificial joints may result in bone damage, either degradation of the interior of the bone or cracking or breaking of the bone, etc. The present invention provides artificial joint members which may be used to cover the damaged part of the bone and extend back to undamaged bone, and thereby provide an alternative to simply fusing the bones together. Additionally, some injuries to the joint and surrounding bones 10, 14 may break or crack the bones in a manner which prevents installation of a prior art artificial joint, such as where cracks or breaks do not leave sufficient strength in the bone for drilling out the bone and installing a prior art artificial joint.

The artificial joint of the present invention is thus advantageous for several reasons. The artificial joint does not require that the bones be drilled out for installation, and as such does not compromise the strength of the bone and result in high stresses in the area of the artificial joint. This may result in an artificial joint which is stronger and which lasts longer than the prior art artificial joints. Additionally, because it extends around the exterior of the bones, the artificial joint of the present invention may be used in some cases to provide an artificial joint where bone damage may otherwise prevent installation of an artificial joint. Thus, the present joint may be installed when another artificial joint must be removed. The artificial joint members may be made to extend past the damaged bone and be connected to strong bone.

The present artificial joint is also advantageous as it better disperses energy through the bones by attachment to the harder outer surface of the bone and not the generally softer inner surface of the bone. The artificial joint also provides ease of surgical placement, as the prior art artificial joints require the proper alignment and drilling of a hole into the bone, where the inventive artificial joint is attached to and aligned by the exterior of the bone.

In addition to the above, the artificial toe joint of the present invention also provides the ability to correct angular deviations, first ray length, as well as providing a resurfacing technique for damaged bones in the toe. Each of these individually provides an improved artificial toe joint and collectively provide a substantial improvement in both technique and ultimate function of the joint.

The artificial toe joint may also provide an easier recovery and surgical procedure. As altering or replacing the sesamoids and cutting or altering their associated tendon would cause further healing and surgical effort, the artificial toe joint of the present invention may provide several reduced healing and surgical effort advantages. By using the existing sesamoid and tendon structure, the patient comfort may increase from before the surgery. Similarly, the surgeon may require less time, less surgical implants and less effort if the bottom structures of the toe may be left alone. The surgeon may not be required to reattach or cut tendons or align structures for the current or replacement sesamoid bones.

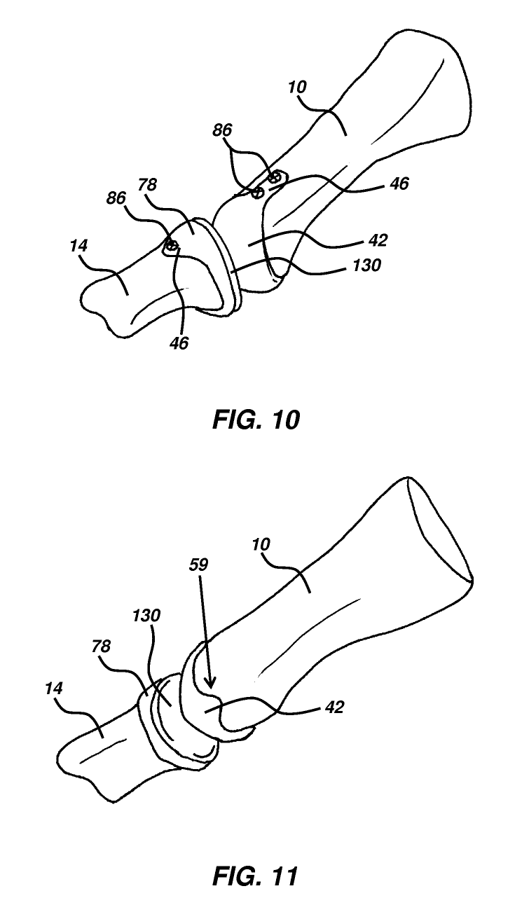

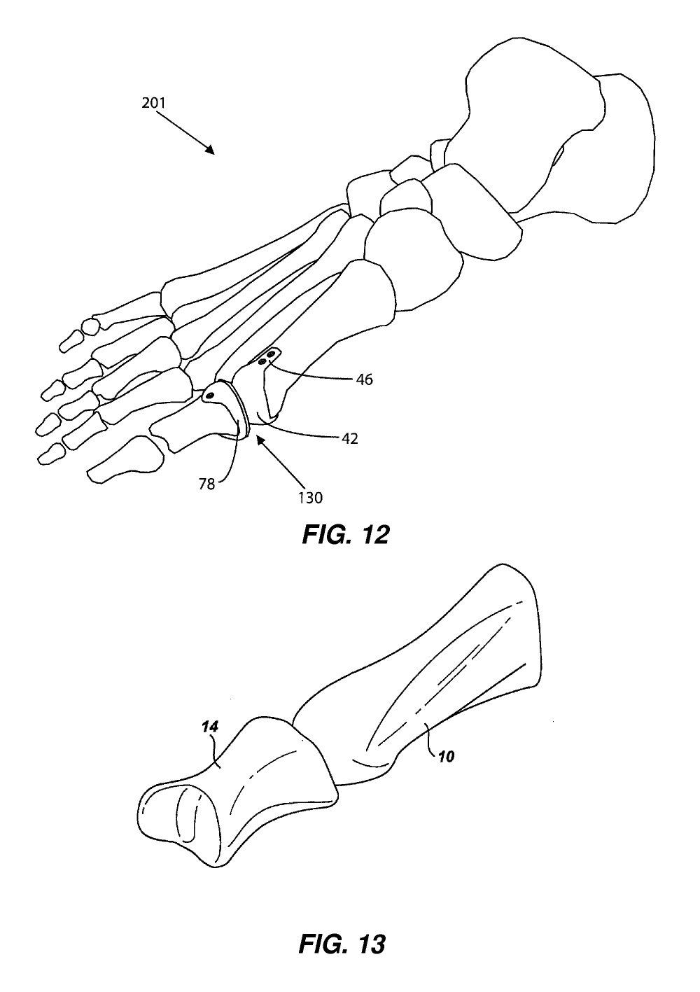

Turning now to FIGS. 10-12, an example of placement of the artificial toe joint members of FIG. 1 are shown. In FIGS. 10 and 11, close-ups of the joint are shown, including positioning of the arms 46 of joint members 42 and 78 extending generally opposite of one another (i.e. one generally proximally and the other generally distally). In FIG. 12, the joint is shown in the larger context of a foot 201. Artificial joint member 42 may ride against or have a complementary rotational association with an insert 130 held in place by a retaining structure in the artificial joint member 78 and/or by artificial joint member 42. Artificial joint member 42 may have a convex external surface configured to mate with a concave surface of artificial joint member 78 (either the metal or other structure which engages the phalangeal bone, or an insert disposed thereon). In FIG. 11, a bottom side wall 59 is formed in a way to minimize impact on the sesamoid bones by avoiding the sesamoid depressions.

Turning now to FIGS. 13 through 15, there is shown the process for preparing metatarsal bone and proximal phalanx bone for installation of a joint made in accordance with the present invention. FIG. 13 shows a perspective view of the metatarsal bone 10 and the proximal phalanx 14. As mentioned regarding FIG. 1, the end of the metatarsal bone is generally convex, while the adjacent end of the proximal phalanx is somewhat concave.

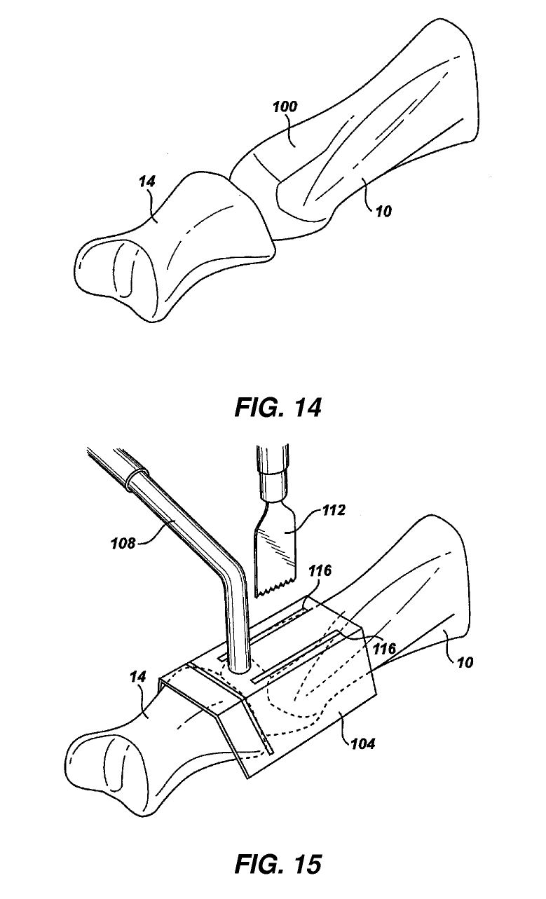

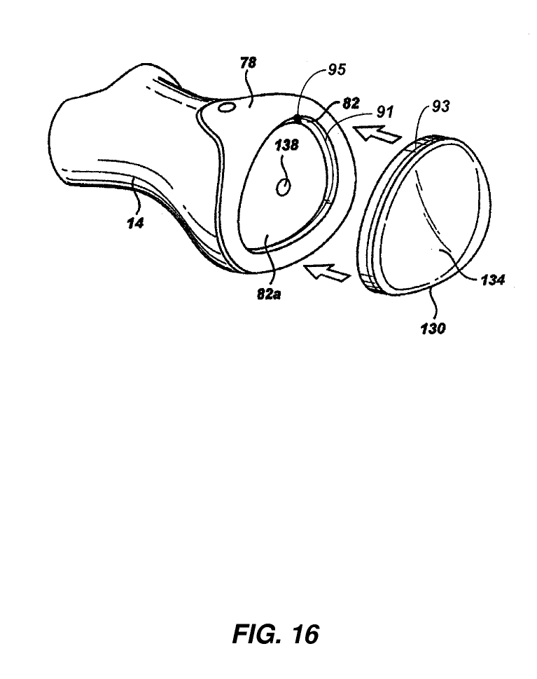

The first step in the process is generally to make a dorsal cut, as indicated at 100 in FIG. 14. The dorsal cut can provide both a surface for ultimate use by the implant, as well as a point of reference for use by a template 104, as shown in FIG. 15. The template 104 may be placed on the metatarsal bone 10 and held in place by a handle 108. An oscillating blade 112 may then be advanced through holes 116 in the template 104 to shape the sides of the metatarsal bone.

It should be noted that the cuts in some cases may occur on just the top and sides of the bone. This may be done, for example, when the end of the bone has already deteriorated to the point that there is no need to cut away bone to position the exterior end of the artificial joint member (42 or 78) at the location where the end of the bone would be in a healthy joint. By cutting on the top and sides of the bone, the procedure also may minimally interfere with the sesamoid bones and related tendons. Indeed, the template may contain guides that prevent the blade from reaching the sesamoid bones or associated tendons.

FIG. 16 shows an exploded view of the proximal phalanx bone with an implant (artificial joint member 78) disposed thereon. As was mentioned previously, the artificial joint member will generally have a recess 82. The recess 82 may be configured to receive the implant 42 attached to the metatarsal bone. In accordance to one aspect of the invention, the recess is configured to receive a cushion or insert 130 which may be disposed between the two. If desired, the artificial joint member 78 may have a shaped recess configured to receive and hold the insert 130.

If desired, the insert 130 can be provided with a structure which assists in alignment of the implants of the artificial joint. More specifically, the insert 130 may include a concave face 134 configured to receive the convex rounded end 62 of the implant 42 attached to the metatarsal bone (FIGS. 3 and 4).

The insert 130 may be made from a variety of biocompatible materials, such as silicone, certain foams, plastics, other polymers, etc. Additionally, the insert may be somewhat flexible, both to provide some cushioning and to facilitate placement in the artificial joint member 78 as explained below.

The artificial joint member 78 may also include a generally flat bottomed recess 82a with a detent 138 formed therein. The recess 82a and detent 138 can help hold the insert 130 in place, thereby providing cushioning in the joint and ensuring that the insert is positioned in a desired orientation relative to the convex surface of the artificial joint member 42 of the metatarsal bone. The insert 130 also provides the advantage that different thicknesses of inserts can be used to adjust for metatarsal length variations. Additionally, the shape of the insert can also be used to correct the angle of the toe relative to the foot. Thus, not only is the doctor able to create an artificial toe joint which more closely matches the normal anatomical structure, he or she can ensure the proper spacing of the metatarsal and proximal phalanx bones to thereby enable the tendons and other structures associated with the joint to operate properly.

The insert 130 may be held in place in the recess 82 by a tongue and groove arrangement with an annular tongue or rib 91 on the artificial joint member 78 engaging a groove 93 on the insert. Other holding structures such as a set screw, adhesive, etc., may also be used.

One significant advantage of the present invention is that the insert 130 allows the physician to use the insert to adjust the length of the toe caused by, for example, brachymetatarsia (a short metatarsal bone). This may be accomplished by selecting an insert 130 which is thicker than normal Likewise, the physician can adjust the angular orientation of the phalangeal bone relative to the metatarsal bone. This may be accomplished by selecting an insert which is shaped on the proximal side 134. Thus, the physician can both provide improved motion in the joint being replaced and correct other problems with the toe such as varus and valgus deformities by increasing medial and lateral thickness of the disk.

To facilitate such corrections, the artificial joint member 78 may include a small channel 95 or nook which is left to enable the physician to insert a tool and remove the insert. This facilitates the replacement of one insert with another which will give the joint a more desirable engagement to normalize function of the toe. It also allows for replacement of the insert 130 if such were ever to become damaged without replacing the remainder of the artificial joint members 42 and 78.

During a surgical procedure, a physician may place and insert and align the joint. If the joint is not aligned properly or if the tendons suggest that the overall toe is too short or the toe is misaligned, the physician can remove the insert and place a new insert which compensates the misalignment or lack of length.

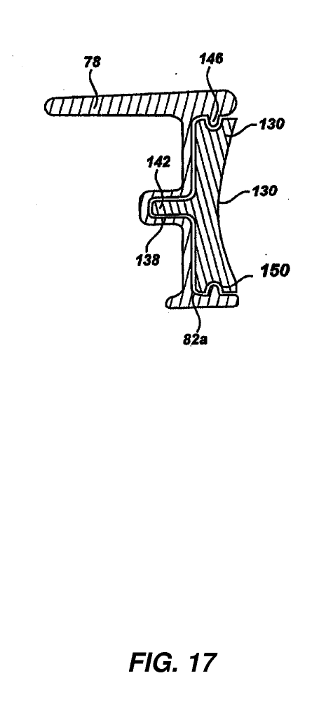

FIG. 17 shows a cross-sectional view of the artificial joint member 78, and the insert 130. The insert 130 may include a projection 142 disposed opposite the concave face 134 to help seat the insert in a projection 138 extending from the recess 82a of the artificial joint member 78. (The relative size of the projection 142 and recess 82a are likely to be smaller or larger proportionally than that shown in FIG. 17 depending on how deeply the physician wants to extend into the phalangeal bone.) Additionally, the recess 82a may include a generally annular rib 146 which is designed to project into a generally annular groove 150 in the side of the insert 130. (It will be appreciated that the annular rib 146 could be replaced with a projection and the annular groove 150 with a detent or other similar structures or the annular rib 146 could be replaced with an annular groove and the annular groove 150 replaced with an annular rib. FIG. 17 can be interpreted to show such structures.) This helps to hold the insert 130 in place and leaves a proximal phalanx with an end which more closely resembles the original anatomical structure.

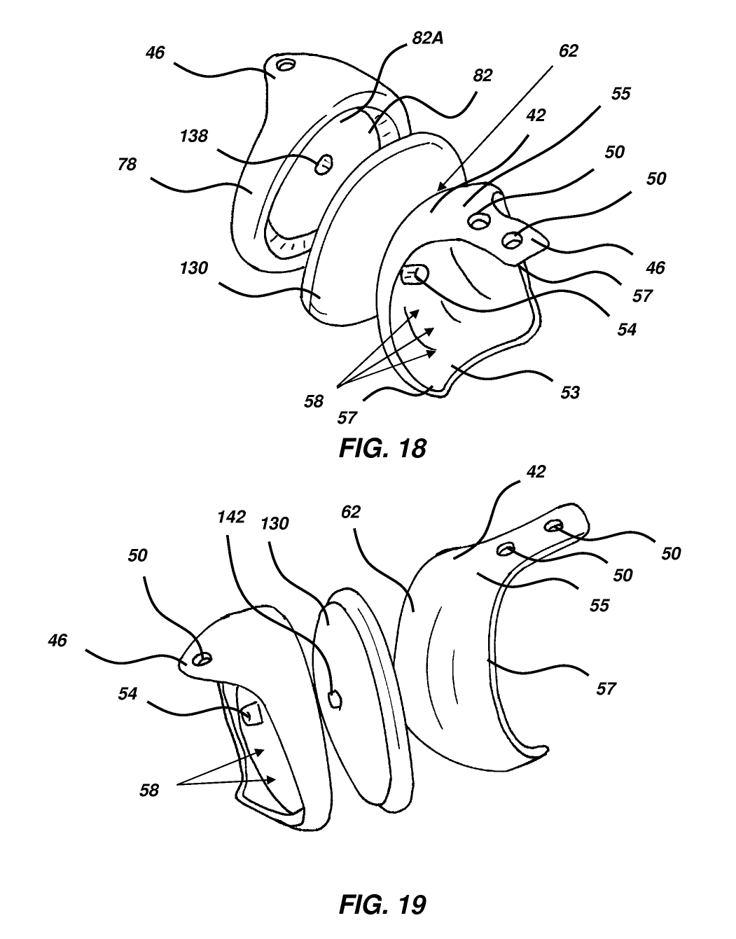

Turning now to FIGS. 18 and 19, exploded views of the artificial joint with an implant are shown. Artificial joint member 42 may rest against the insert 130 that is placed within the other artificial joint member 78 and may effectively be part of artificial joint member 78. The angle of the joint may be adjusted by adjusting the thickness of portions of the insert 130. By having portions of the insert 130 thicker than other portions of the insert, the artificial joint axis can be manipulated. Thus the engagement of the phalangeal bone may be moved down if a bottom portion is thicker than the top portion of the insert 130. The joint may be shifted left, if a right portion of the insert 130 is thicker than a left portion or vice-versa.

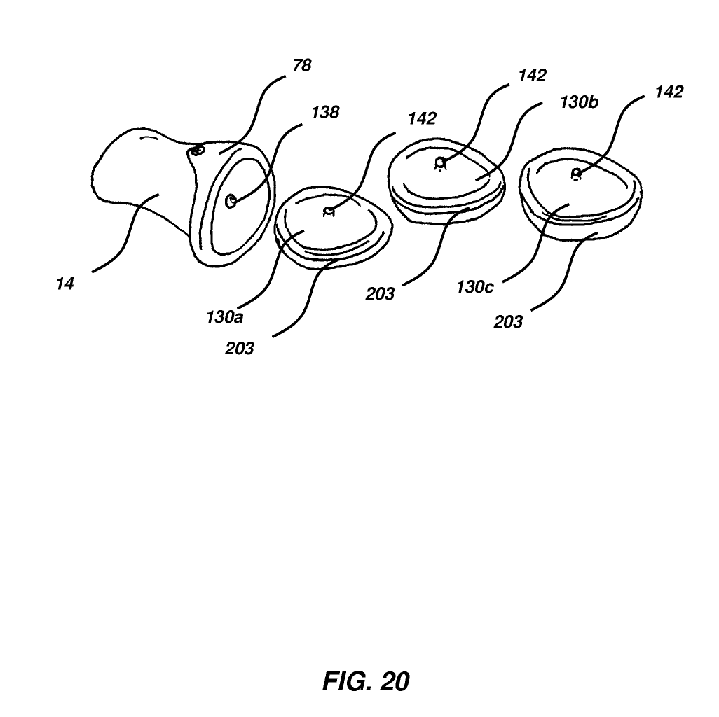

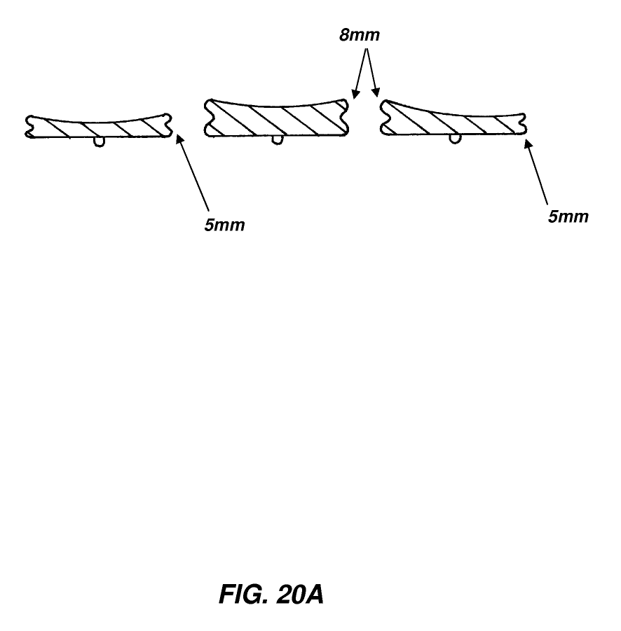

Turning now to FIG. 20, there is shown a proximal phalanx bone with an implant (artificial joint member 78) disposed thereon and a plurality of inserts 130A, 130B and 130C which have a sidewall 230 having different thicknesses. The inserts 130 may be customized to the patient to correct joint deformities. For example, an insert may be tapered to adjust the direction of a toe. If the toe is required to point medially (more inward), an insert having a greater the thickness on the lateral or outward portion than the medial or inward portion of the insert may be selected. If the joint is prone to separation, the insert may be shaped or sculpted to aid in preventing separation. In one embodiment the side walls of the insert are further extended around the adjoining artificial joint member. In another embodiment, a sidewall is extended to aid in preventing the expected separation.

By way of example a standard disk for use in the insert 78 may be up to 10 mm and may typically be about 5 mm in thickness with opposing sidewalls being substantially the same thickness. A person having a typical toe length and without need for angular correction may thus receive a disk of 5 mm mounted in the insert 78.

If, in contrast, a person has an unusually short phalange bone, the physician inserting the artificial toe joint may select a disk 130b which is 10 mm or even 15 mm thick depending on the undersized nature of the bone. By doing so, the physician can readily adjust the toe length to give the user a more natural toe configuration after the surgery.

Likewise, the insert can also be used to correct Hallux Varus and Valgus. Hallux Varus is where the big toe deviates toward the midline of the body and away from the second toe. Hallux Valgus, more commonly known as a bunion, is where the big toe deviates laterally away from the midline of the body and toward the second toe. Either condition can affect the ability of a person to walk normally and each can be corrected in accordance with the present invention.

An angular measurement used to determine bunion severity is known as intermetatarsal angle with a normal range of 8 to 10 degrees. This angle may be obtained by bisecting the long axis of first metatarsal drawing a line down the center of first metatarsal from the head to the base. A second line is taken in a like manner however using the second metatarsal. The bisection of these two lines creates an angle known as the intermetatarsal angle and may normally up to about 8 to 10 degrees. The angle size has a direct proportion to aggressiveness and complexity of surgery. An angle of greater than 11 usually requires surgical correction. (A second angle measurement which can also be addressed is known as the Hallux Abductus angle. It is the angular measurement between the longitudinal bisection of the 1st metatarsal and the proximal phalanx. The normal Hallux Abductus angle is generally less than 10 degrees.)

Traditional methods of treating Hallux Valgus and Varus may include either cutting out of portion of the toe or driving a wedge into the toe (typically the phalangeal bone) to reshape the toe and change its overall orientation. Obviously, either can be traumatic and require extensive recuperation.

The inserts 130A, 130B or 130C, may allow for hallux limitus correction (loss of motion) as well as Hallux valgus or Hallux varus correction secondary to the disc component. It has been determined that a 1 mm increase in disc thickness may result in a decrease of 1.5 intermetatarsal angle. Thus, if a person has a bunion in which the toe is 10 degrees outside of the normal range, this may be fixed by having the insert having a lateral side which is 6-7 mm thicker than the opposing side. This difference in thickness can change the interface between the insert and the implant on the metatarsal bone, thereby correcting the defect.

It will be appreciated that the change in angular orientation achieved by different thicknesses may depend in part of the size of an insert. The smaller the insert, the greater the angular change which will be created by each millimeter of increase on one side of the insert.

In addition to correcting angular deformation, it is believed that the present invention may substantially reduce recuperation time. It is believed that the procedure of the current invention is likely to return the patient to an ambulatory status in 14 days versus the 4 to 6 weeks required for some other surgical procedures.

The present invention may also be advantageous in correcting hammer toes. In some treatments, hammer toes are corrected by removing the head of the proximal phalange and the base of the intermediate phalange then fusing the two bones. The resections reduce length allowing the toes to become straight, however, motion is lost. With the implant of the present invention, resection can occur without reaming the bone and the implant can be placed to correct the hammer toe without loss of motion.

By allowing the surgeon to choose an insert 130A, 130B, 130C designed for a modification to the joint, the physician may custom design the joint to improve the range of motion and orientation, thereby increasing the likelihood of improved function. In one embodiment the surgeon may choose a size of artificial joint member 42 and artificial joint member 78 based on the size, such as a radius, of a metatarsal bone 10 and a phalangeal bone 14. The joint direction and distance may then be customized by selecting an appropriate insert 130A, 130B or 130C. The insert 130A may have a simple, even contour on its face to allow a normally oriented metatarsal and phalangeal bone to move with respect to one another. In the alternative, the insert 130B may be thicker to accommodate for a short metatarsal or phalangeal bone, or the insert 130C may have tapered sidewalls 203 to correct joint anomalies, such as direction, or to adjust joint length. In some cases it may be useful to have an insert sidewall 203 to extend further to prevent the joint from slipping out.

The inserts may be selected from pre-fabricated versions, molded on site or altered on site. In one embodiment, the physician may have a standard set of inserts from which he or she may choose a desired angle and joint length. In another embodiment, the physician may have one or more inserts that may be shaved, sanded, molded (cast or melted and reformed) or otherwise formed to accommodate different angles and/or joint lengths. In another embodiment, the physician may have an adjustable mold that may create inserts of different thicknesses and/or shaped to provide different angles to the joint.

FIG. 20A shows cross-sectional views of the inserts 130A, 130B, 130C from FIG. 20. The cross-sectional view demonstrates the differences in thickness, as well as modification to the concave surface which engages the artificial joint member for the metatarsal bone. By selecting an appropriate insert, the physician can customize the artificial joint and may even improve functionality of the joint above the natural joint of the patient. For example, if the digit is normal, the surgeon may select insert 130A which has a desired thickness, typically about 5 mm and mount it in the implant 78 (FIG. 20) prior to aligning the bones. If the toe is short, the physician may select insert 130B which is thicker than normal (8 mm as shown) or an even thicker insert (not shown) to add length to the toe to allow for proper use of the tendons.

If the joint being replaced deviates to the medial or inside of the foot, the surgeon may select and insert 130C which is thicker or wider on the medial side. This will urge the digit away from the medial or inside of the foot, thereby encouraging the toe toward the second toe and into a more natural position.

If the joint being replaced deviates to the lateral or outside of the foot, i.e. a bunion, an insert having a minor image of 130C could be used so that the insert 130C is thicker on the lateral side, thereby directing the toe medially and away from the second toe. Obviously, the physician may preferably have access to a number of different inserts having different thicknesses in different parts to correct angular irregularities. For example, insert 130C may provide for approximately 4-5 degrees of correction and another insert having an even greater thickness on the left side would provide even greater correction. The amount of correction provided may depend, at least in part, on a function of the change in thickness and other width of the insert, with a smaller insert (i.e. which could be used between phalangeal bones) providing more angular change per additional millimeter of thickness than a larger insert used for a metatarsal/phalangeal joint.

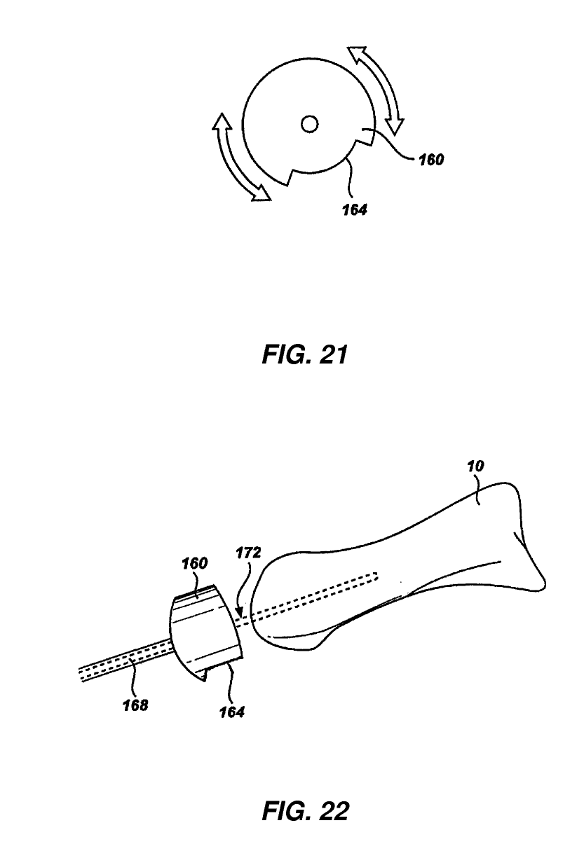

FIG. 21 shows a bit 160 which can be used to improve implant surface preparation. Rather than using the template system described in FIGS. 13-15, the bit 160 is attached to a drill (not shown). The bit 160 is a concave oscillating bit and includes a notch 164 for the sesamoid bones. The notch 164 prevents damage to the sesamoid depressions in the metatarsal bone and interference with the tendon of the flexor hallucis brevis. Thus, one advantage is that the surgeon may not have to reattach the tendon, nor worry about the sesamoid bones placement. As sesamoid bone sizes and shapes may differ per person, the surgeon may also avoid having to customize the implant for the sesamoid bones and flexor hallucis brevis. Or, the patient may not have to adjust to sesamoid implants, which may feel different than the original sesamoid bones.

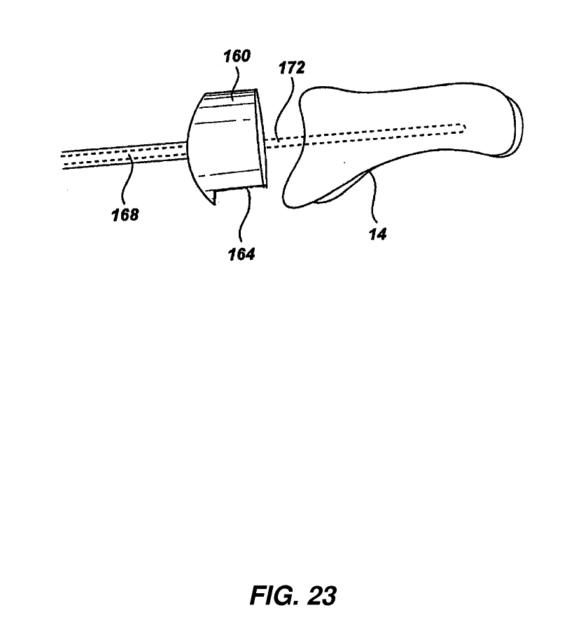

The drill may include a cannulated shaft 168 with a K-wire 172 extending therefrom. The K-wire is used to align the bit 160 on either the metatarsal or proximal phalanx bone depending on which piece is being worked. Thus, FIG. 22 shows the bit 160 being advanced on the metatarsal, and FIG. 23 shows the bit being advanced on the proximal phalanx.

The bit 160 allows the ends of the respective bones to be reshaped for improved mounting of the artificial joint members and allows a remaining bone structure which is more anatomically correct with potentially less interference with existing structures, such as the sesamoid bones. Once the bit 160 has prepared the bones, the artificial joint members can be attached, thereby creating a new joint.

Because the artificial joint engages a much greater surface area of the bones, less stress is placed on the bones and the risk of further damage is decreased. Additionally, if the insert 130 were to fail for some reason, it can simply be replaced without further damage to the bones. This is in sharp contrast to some prior artificial toe joints which generally result in fusion of the bones when they fail.

While the present invention has been discussed in detail with respect to a toe joint, it will be appreciated that aspects of the present invention could be used for other anatomical structures as well. For example, the present invention could be used to repair a damaged finger joint or to correct the angular orientation between bones, such as in a misdirected finger.

There is thus disclosed an improved artificial toe joint. It will be appreciated that numerous changes may be made to the present invention without departing from the scope of the claims.

What is claimed is:

1. An artificial joint comprising:
a first artificial joint member having a first side and a second side and a support member extending from the second side;
a second artificial joint member having a first side and a second side and a support member extending from the second side; and
wherein the first side of the first artificial joint member and the first side of the second artificial joint member comprise structures which engage one another to form a movable joint;
wherein the support member of the first artificial joint member extends away from the joint along the outside surface of bone when installed.

2. The artificial joint of claim 1, further comprising a metatarsal bone having an end and depressions adjacent the end for receiving sesamoid bones and wherein the first artificial joint member is attached to cover the end of the metatarsal bone without extending into the depressions.

3. The artificial joint of claim 2, wherein the second artificial joint is attached to a phalangeal bone having an end, the phalangeal bone having a tendon of the flexor hallucis brevis attached thereto, and wherein the second artificial joint is attached so as to cover the end of the phalangeal bone without interfering with the attachment of the tendon to the phalangeal bone.

4. The artificial joint of claim 1, wherein the first artificial joint member has a side wall which is formed to wrap substantially around depressions within a metatarsal bone when installed.

5. The artificial joint of claim 1, wherein the first artificial joint member has a side wall which does not extend within depressions within a metatarsal bone when installed.

6. The artificial joint of claim 55, wherein the support member is a collar.

7. The artificial joint of claim 6, wherein the collar has an opening positioned around the depressions within the metatarsal bone.

8. The artificial joint of claim 5, wherein the support member is an arm.

9. The artificial joint of claim 8, wherein the arm is opposite the side wall.

10. The artificial joint of claim 1, wherein the second artificial joint member further comprises a second support member.

11. A method for forming an artificial toe joint comprising:
selecting a first artificial joint member and attaching the first artificial joint member to an end of a metatarsal bone adjacent a phalangeal bone, the metatarsal bone having depressions formed therein for receiving sesamoid bones, wherein the first artificial joint member is positioned to cover an end of the metatarsal bone and form a generally convex joint surface without interfering with sesamoid bones received in depressions in the metatarsal bone.

12. The method according to claim 11, wherein the method comprises selecting an artificial metatarsal joint member having a first side for forming the generally convex joint surface and a support member extending away from the generally convex joint surface and attaching the support member to the exterior of the bone remote from the end of the bone.

13. The method according to claim 11, further comprising a second artificial joint member attached to an end of a phalange bone adjacent the artificial joint member attached to the metatarsal bone, the second artificial joint member covering an end of the phalangeal bone without interfering with attachment of a tendon of the flexor hallucis brevis to the phalangeal bone.

14. The method according to claim 13, wherein the second artificial joint member has a support arm extending away from the metatarsal artificial joint member which is attached to the exterior of the phalangeal bone distal from the end of the phalangeal bone.

15. The method according to claim 13, further comprising disposing an insert disposed in the second artificial joint member so as to receive the metatarsal artificial joint member.

16. The method according to claim 15, wherein the method comprising selecting an insert having a desired thickness to adjust the length of the toe to a desired length.

17. The method according to claim 15, wherein the method comprising selecting an insert having a concave portion at a desired location of the insert so as to correct an angular orientation between the metatarsal bone and the phalangeal bone.

18. The method of claim 13, wherein the method comprises shaping the end of the phalangeal bone with a generally circular saw blade having a cut-out formed therein so as to reshape a portion of the end without cutting into bone structure attached to a tendon of the flexor hallucis brevis.

19. The method of claim 11, wherein the method comprises shaping the end of the metatarsal bone with a generally circular saw blade having a cut-out formed therein so as to reshape a portion of the end without cutting into bone structure adjacent the depressions in the metatarsal bone which receive sesamoid bones.

20. An artificial toe joint comprising:
a first artificial joint member further comprising a top side, lateral sides and a bottom side, a support member extending from a periphery of at least one of the top and sides, the support member being configured for attachment to the exterior of a bone; a first side of the artificial joint member being generally convex and a second side being generally concave so as to receive and cover an end of a bone, the bottom side of the artificial joint member having a nook formed therein shaped to receive a tendon passing along the bone.

21. The artificial toe joint of claim 20, further comprising a second artificial joint member configured with a complementary surface to the first artificial metatarsal joint member, the second artificial joint member further comprising a support member extending from a periphery of the second artificial joint member for attachment to the exterior of the bone.

22. The artificial toe joint of claim 21, wherein the second artificial joint member further comprises an insert removably attached to the remainder of the second artificial joint member, the insert having a generally concave surface for receiving the first artificial joint member.

23. The artificial toe joint of claim 20, further comprising an insert placed between the artificial metatarsal joint member and the artificial phalange joint member.

24. An artificial joint comprising:
a first artificial joint member having a generally concave recess for receiving an end portion of a bone, and at least one appendage extending from a periphery about the recess rearwardly so as to engage an outside surface of a bone, the first artificial joint member having a convex surface extending on a side opposite the at least one appendage for engaging a second artificial joint member;
a second artificial joint member having a generally concave recess for receiving an end portion of a second bone, and at least one appendage extending from a periphery about the recess so as to engage an outside surface of a bone, the second artificial joint member having a second recess opposite the first recess for general alignment with the convex surface of the first artificial joint member.

25. The artificial joint of claim 24, wherein the joint further comprises a removable insert disposed in the second recess of the second artificial joint member, the removable insert having a concave surface for receiving the convex surface of the first artificial joint member.

26. The artificial joint of claim 25, wherein the first artificial joint member and the second artificial joint member are aligned such that the at least one appendage of each artificial joint member extend generally opposite one another.

27. The artificial joint member of claim 24, wherein the at least one appendage of the first artificial joint member comprises at least one arm.

28. The artificial joint member of claim 24, wherein the at least one appendage of the first artificial joint member comprises a collar.

29. The artificial joint member of claim 24, wherein the at least one appendage of the second artificial joint member comprises at least one arm.

30. The artificial joint member of claim 26, wherein the at least one appendage of the second artificial joint member comprises a collar.
